(19) United States
(12) Patent Application Publication
(10) Pub. No.: US 2026/0345863 A1
(43) Pub. Date: Nov. 5, 2026

(54) SUTURELESS, SELF-RETAINING BIO-TISSUE INSERT FOR THE OCULAR SURFACE AND METHODS OF USE

(71) Applicant: Interstat, LLC, White Plains, NY (US)

(72) Inventors: Ravinder D. Pamnani, White Plains, NY (US); Phillip Ianchulev, White Plains, NY (US); Tsontcho Ianchulev, White Plains, NY (US)

(21) Appl. No.: 19/486,786

(22) PCT Filed: May 29, 2024
(86) PCT No.: PCT/US2024/031450

§ 371 Date: Nov. 21, 2025

Related U.S. Application Data
(60) Provisional application No. 63/469,469, filed on May 29, 2023.

(51) Int. Cl. A61L 27/36

(52) U.S. Cl. CPC A61L 27/3604; A61L 27/3641; A61L 2430/16

(57) ABSTRACT

An amniotic membrane insert configured for self-retaining, sutureless, clear-cornea application to an ocular surface. The insert has an outer edge and an inner edge inside the outer edge. The inner edge defines an opening sized to extend around a circumference of a cornea. Related devices, systems, and methods are provided.

### CROSS-REFERENCE TO RELATED APPLICATIONS

This application claims the benefit of priority under 35 U.S.C. § 119(e) to U.S. Provisional Application Ser. No. 63/469,469, filed May 29, 2023. The disclosure is incorporated by reference herein in its entirety.

### BACKGROUND

The chorioamniotic membrane separates the fetus from the mother's endometrium in mammals and includes the amniotic membrane, or amnion, and the chorionic membrane, or chorion. The amniotic membrane is the innermost layer of the chorioamniotic membrane. The amniotic membrane is a thin, transparent tissue that also has several layers, including epithelial layer, a basement membrane, a compact layer, and a fibroblast layer. The amniotic membrane is rich in collagen and various bioactive factors having properties that contribute to the healing process.

The amniotic membrane is obtained from the placenta after childbirth and is known for its use in the treatment of burns and wounds. Amniotic membrane has been used on the ocular surface for its healing and regenerative effects. Key amniotic factors such as Prostaglandin E2 (PGE2), Growth Differentiation Factor 11 (GDF11), Thrombospondin-1, and WNT4 have been identified as major modulators of the healing, anti-inflammatory and immune protective properties of amniotic membrane (see J. Trans. Med. 12, 260(2014 ); Cell Res. 24(12): 1381-1382 (2014); J. Ocul. Pharmacol. Ther. 31(7): 376-385 (2015); Clin. Ophthalmol. 12:677-681 (2018); J. Ophthalmol. 2017; 2017:6404918. doi: 10.1155/2017/6404918).

One of the main challenges associated with the clinical use of amniotic tissue on the ocular surface is the difficulty of attaching it non-invasively and durably in a way that does not cause irritation, discomfort and pain in the eye—as it is well established that the ocular/corneal surface is one of the most sensitive tissues in the human body.

### SUMMARY

In an aspect, provided is an amniotic membrane insert configured for self-retaining, sutureless, clear-cornea application to an ocular surface. The insert has an outer edge and an inner edge inside the outer edge. The inner edge defines an opening sized to extend around a circumference of a cornea.

The outer edge can have a shape that is circular, oval, or elliptical. The opening can be at least 9 mm in diameter. The outer edge can have a diameter that is no greater than 33 mm and at least about 12 mm. The insert can have a width between the outer edge and the inner edge that is at least 100 microns and no greater than 8 mm.

The insert can be formed solely of tissue without any retention feature or other structure. Alternatively, the insert can incorporate a retention feature separate from the insert that is configured to provide structural stability to the insert. The retention feature can be formed of a biodegradable or non-biodegradable material. The retention feature can include a bandage or standard corneal contact lens. The retention feature can have an outer perimeter sized to be received within the opening of the insert. The inner edge of the insert can have a diameter that is 0.1 mm-2.0 mm greater than a diameter of the outer perimeter of the retention feature. The insert can have a width between the outer edge and the inner edge, wherein the thickness is conforming to the retention feature. The width of the insert can be less than 4 mm. The width of the insert can be less than a thickness of the retention feature.

In an interrelated aspect, provided is an insert made of amniotic membrane that is configured for self-retaining, sutureless, clear-cornea application to an ocular surface. The insert has an outer edge, an inner edge, a first free end, and a second free end opposite the first free end. A length of the insert between the first free end and the second free end and a width of the insert between the outer edge and the inner edge is sized to be received within the cul-de-sac of an eyelid.

The eyelid can an upper eyelid or a lower eyelid. The length of the insert can be no greater than 33 mm, the width of the insert can be no greater than 100 mm, and a thickness of the amniotic membrane can be no greater than 5 mm. The insert can be formed solely of tissue without any retention feature or other structure. Alternatively, the insert can incorporate a retention feature separate from the insert and configured to provide structural stability to the insert. The retention feature can be formed of a biodegradable or non-biodegradable material. The amniotic membrane can include amnion membrane. The amniotic membrane can include amnion membrane and chorion layer.

In an interrelated aspect, provided is a method of administering biological factors to an eye including applying amniotic membrane shaped to have an outer edge and an inner edge inside the outer edge, the inner edge defining an opening; positioning the amniotic membrane onto an anterior surface of the eye so that the opening surrounds a circumference of a cornea of the eye and the inner edge does not contact the cornea; and releasing biological factors from the amniotic membrane to the eye.

In an interrelated aspect, provided is a method of minimally-modifying amniotic membrane tissue into an annular shape configured to be received on an anterior surface of the eye that avoids contacting the cornea including cutting amniotic membrane tissue into an elongate strip, the strip having a length between a first free end and a second free end, a width across the length, and a thickness; and connecting the first free end and the second free end to form a closed loop, the closed loop defining a central opening sized to surround a circumference of the cornea.

In an interrelated aspect, provided is a method of minimally-modifying amniotic membrane tissue for treatment of an eye. The method includes cutting the amniotic membrane tissue into an annulus, the annulus having an outer edge, an inner edge inside the outer edge that defines a central opening sized to surround a circumference of the cornea without contacting the cornea.

In an interrelated aspect, provided is an insert of amniotic membrane coupled to a punctal plug, the punctal plug sized and shaped to insert at least in part within a punctum of an eye to position the amniotic membrane in contact with a surface of the eye.

The amniotic membrane can be shaped into an elongate strip or disk. The punctal plug can be solid so as to occlude the punctum and reduce drainage of fluid out of the eye.

Upon positioning the insert in the eye, the amniotic membrane can be positioned under a lid of the eye. The amniotic membrane can be positionable within the cul-de-sac of the eye. The punctal plug can include a proximal collar, a distal tapered region, and a body extending between the collar and the tapered region. The amniotic membrane can be coupled to the collar of the punctal plug by a tissue connector. The insert can further include second punctal plug coupled to the amniotic membrane. The punctal plug and/or the tissue connector can be amniotic membrane.

In an interrelated aspect, provided is an insert of amniotic membrane coupled on a first end to a first punctal plug and coupled on a second end to a second punctal plug. The first punctal plug is sized and shaped to insert at least in part within a first punctum of an eye. The second punctal plug is sized and shaped to insert at least in part within a second punctum of the eye to position the amniotic membrane spanning between the first and the second punctal plugs in contact with a medial anterior surface of the eye. The first and second punctal plugs can be amniotic membrane.

The amniotic membrane can be shaped into an elongate strip or disk. At least one of the first and second punctal plugs can be solid to occlude the punctum and reduce drainage of fluid out of the eye.

Other features and advantages should be apparent from the following description of various implementations, which illustrate, by way of example, the principles of the invention.

### BRIEF DESCRIPTION OF THE DRAWINGS

These and other aspects will now be described in detail with reference to the following drawings. Generally, the figures are not to scale in absolute terms or comparatively but are intended to be illustrative. Also, relative placement of features and elements may be modified for the purpose of illustrative clarity.

FIG. 1 shows an implementation of a bio-tissue insert;

FIG. 2 illustrates the bio-tissue insert of FIG. 1 positioned on an ocular surface;

FIG. 3 illustrates another implementation of a bio-tissue insert positioned on an ocular surface;

FIG. 4A illustrates another implementation of a bio-tissue insert in an initial cut form factor;

FIG. 4B illustrates the bio-tissue insert of FIG. 4A after fusing into a closed loop having a circular shape;

FIG. 5A illustrates another implementation of a bio-tissue insert in an initial cut form factor;

FIG. 5B illustrates the bio-tissue insert of FIG. 5A after fusing into a closed loop having a polygonal shape;

FIG. 5C illustrates another implementation of a bio-tissue insert in an initial cut form factor;

FIGS. 6A-6F illustrate cross-sectional shapes of the bio-tissue insert of FIG. 1 taken along line A-A;

FIG. 7 illustrates a bio-tissue insert coupled to a retention feature;

FIG. 8A illustrates another implementation of a bio-tissue insert coupled to a retention feature;

FIG. 8B illustrates an anterior surface of an eye and the lacrimal system;

FIG. 8C is a schematic of the insert and retention feature of the FIG. 8A fixed within the puncta of the eye.

It should be appreciated that the drawings are for example only and are not meant to be to scale. It is to be understood that devices described herein may include features not necessarily depicted in each figure.

### DETAILED DESCRIPTION

Described herein are bio-tissue inserts, formed for example of amniotic membrane allograft or xenograft, for use on the ocular surface to treat inflammatory, neovascular or other ophthalmic diseases and processes of the eye. The bio-tissue inserts are sutureless and self-retaining.

Therapeutic approaches for delivery of amniotic products to the ocular surface are known. Amniotic membrane corneal shield/bandage lens products are commercially available, such as PROKERA (BioTissue, Inc., Miami, FL), AMBIODISK (Corza Medical, Inc., Westwood, MA) and AMNIOEXCEL (Integra Lifesciences, Inc., San Diego, CA). These corneal shields are exclusively applied on the cornea in the central 10-12 mm of the eye. The shields are circular amniotic tissue discs often attached to the corneal surface using additional retention hardware, such as a bandage contact lens designed to improve stability and retention over the cornea. The amniotic membrane corneal shield typically dissolves after 7-10 days application to the eye as its therapeutic diffusible factors aide the healing of the corneal surface.

A key challenge to the current ocular use of amniotic membrane is that the amniotic corneal disc is positioned over the cornea and causes significant intolerance and pain. This is because the cornea is the most sensitive part of the eye, as it contains unmyelinated nerve endings and the density of nerve endings in the corneal epithelium is 400 times that of the epidermis of the skin (Clinical Anatomy and Physiology of the Visual System (Third Edition), 2012). Additionally, the cornea lacks blood vessels due to the need for transparency, meaning oxygen from the ambient air keeps the cornea healthy. A covering for 7-10 days by an amniotic membrane corneal shield is detrimental to oxygen uptake. In addition, the amniotic membrane is cloudy and optically detrimental to visual axis, which leads to reduced vison and blur.

Amniotic tissue-derived extract in the form of eye drops is another therapeutic approach for providing the healing power of amniotic membrane to an eye. The improvements in ocular surface healing and comfort associated with amniotic membrane transplantation have been attributed, in part, to the diffusible anti-inflammatory properties of the tissue, inhibiting the expression of pro-inflammatory mediators and growth factors. This has been demonstrated in studies of the wound healing applications of amniotic tissue-derived extracts to the ocular surface whereby the amnio cytokines and anti-inflammatory and healing factors (more than 120 of them) were delivered as eye drops without the application of amniotic membrane shield or corneal bandage to the ocular surface (Clinical Ophthalmology 2019:13 887-894). Those cytokines, growth factors, and enzyme inhibitors involved in tissue remodeling (e.g., matrix metalloproteinases) showed dramatic improvement in ocular surface healing and dry eye disease scores. A key challenge with amniotic tissue-derived extracts in the form of eye drops is that it requires daily patient adherence to consistently self-dose the therapy. In one study, adherence to the prescribed dosing regimen in dry eye patients was as low as 10% (Uchino M, et al. Adherence to Eye Drops Usage in Dry Eye Patients and Reasons for Non-Compliance: A Web-Based Survey. J. Clin. Med. 2022 Jan. 12; 11(2):367).

There remains a significant need for a better method and form-factor for amniotic membrane application to the ocular surface for the delivery of therapeutic diffusible amniotic factors, which would not interfere with the visual axis, is associated with minimal or no pain and discomfort, will not dislodge and extrude from the eye, and is not dependent on daily patient adherence.

Described herein are amniotic membrane inserts that include amniotic bio-tissue, with or without additional non-tissue structural hardware, for application to the ocular surface. The inserts are designed to avoid impacting the visual axis and avoid the central corneal surface for optimal non-invasive retention and reduced pain and discomfort. At the same time, the amniotic tissue remains resident on the ocular surface to dissolve over time and deliver its therapeutic diffusible factors.

The term “biologically-derived material” or “bio-tissue” includes naturally-occurring biological materials that are suitable for implantation onto the eye. Biologically-derived material includes a material that is a natural biostructure having a biological arrangement naturally found within a mammalian subject including organs or parts of organs formed of tissues, and tissues formed of materials grouped together according to structure and function. Biologically-derived material includes tissues such as amniotic membrane, chorioamniotic membrane, amnion, or chorion. Biologically-derived material includes tissues such as corneal, scleral, or cartilaginous tissues as well as acellular biomatrix tissue.

Biologically-derived material includes tissue harvested from a donor or the patient, organs, parts of organs, and tissues from a subject including a piece of tissue suitable for transplant including an autograft, allograft, and xenograft material. Biologically-derived material includes naturally-occurring biological material including any material naturally found in the body of a mammal.

Biologically-derived materials can include naturally-occurring biological tissue including any material naturally found in the body of a mammal that is minimally-manipulated or more than minimally-manipulated according to FDA guidance under 21 CFR § 1271.3(f) such that the processing of the biological tissue does not alter the relevant biological characteristics of the tissue (see Regulatory Considerations for Human Cells, Tissues, and Cellular and Tissue-Based Products: Minimal Manipulation and Homologous Use, www.fda.gov/regulatory-information/search-fda-guidance-documents/regulatory-considerations-human-cells-tissues-and-cellular-and-tissue-based-products-minimal).

The biologically-derived material, sometimes referred to herein as bio-tissue or bio-material, which is used to form the insert can vary and can be, for example, chorioamniotic membrane, amniotic membrane, or amnion, and the chorionic membrane, or chorion, and the umbilical cord. Bio-tissue can alternatively or additionally include corneal tissue, scleral tissue, cartilaginous tissue, collagenous tissue, or other firm biologic tissue.

Bio-tissue can include tissue from a mammal, including human and non-human animal tissue.

The inserts described herein can be made of 1% to 100% amniotic tissue with the rest consisting of a bio-degradable or non-biodegradable polymer. The amniotic membrane may consist of amnion only (single layer amnion or dual layer amnion), or may consist of both amnion and chorion layers, or the membrane may consist of umbilical cord tissue. In another embodiment, the membrane sheet thickness may consist of stacked layers of amnion, chorion, or umbilical cord tissues. The amnion has a Young's modulus that is stiffer than chorion (e.g., 21 MPa vs. 2.3 MPa) (see Verbruggen et. al., “Function and failure of the fetal membrane: Modelling the mechanics of the chorion and amnion” PLOS ONE Mar 28, 2017). The amnion provides some heft to the chorion to ensure the dual layer amnion-chorion inserts are user friendly enough for applying the insert to the anterior surface of the eye.

The biologically-derived material can include or be capable of releasing one or more factors of the biologically-derived material for providing treatment of a disease or condition. For example, the material can be a tissue that releases healing factors derived from the tissue that have anti-fibrotic, anti-inflammatory, anti-neovascular effects, or the like for repair and regeneration at the site of implantation or near the site of implantation. The material can be whole amniotic membrane that releases one or more regenerative and anti-fibrotic factors that aid in the control of inflammation and scarring including, but not limited to VEGF (Vascular Endothelial Growth Factor), VEGF-R (VEGF Receptor), ANG1 (Angiopoietin 1), TIMP-1 (Collagenase inhibitor), TIMP-2 (Collagenase Inhibitor), IL-1B (Interleukin 1B), PDGF-AA (Platelet Derived Growth Factor), TGFb3 (Transforming Growth Factor Beta 3), bFGF (Basic Fibroblast Growth Factor), and HGF (Heptocyte Growth Factor), Prostaglandin E2 (PGE2), Growth Differentiation Factor 11 (GDF11), Thrombospondin-1, and WNT4. The amniotic membrane contains both growth promoting and growth inhibiting proteins (see, e.g., Clinical Ophthalmology 2019:13, 887-894).

The biologically-derived material can be amniotic membrane. The amniotic membrane tissue can be derived from amniotic sac of the placenta. The amniotic sac surrounding a fetus includes the amnion and chorion, both derived from the inner layer of the placenta. The amniotic membrane is part of the amnion and includes a layer of epithelial cells, a basement membrane, and an avascular stromal matrix. Amniotic membrane contains pluripotent cells, highly organized collagen, anti-fibrotic and anti-inflammatory cytokines, immune-modulators, growth factors, and matrix proteins and can promote healing in eyes (Murri et. al. Clin. Ophthalmol. (2018) 12:1105-1112). While inserted on the ocular surface, the amniotic membrane can release factors useful for tissue repair and regeneration. The amniotic membrane bio-absorbs after at least about 1 week up to about 3 to 6 months. The inserts formed of amniotic membrane can be placed on the ocular surface for a fixed period of time for at least 1 day up to about 3 to 6 months and then removed before bio-absorption, after at least one bioactive factor has completely or partially diffused into the tear film. The inserts formed of amniotic membrane can be used for delivery of beneficial diffusible factors from the tissue that can be helpful against a variety of conditions. The inserts formed of amniotic membrane can be inserted alone as a primary treatment or in combination with another biotissue implant(s) formed of other materials (e.g., sclera) or with another medical therapy (e.g., eye drops composed of one or more drugs) as an adjunct anti-fibrotic or anti-inflammatory treatment.

The tissue can be freeze-dried, lyophilized membrane, dehydrated, cryopreserved, or otherwise minimally-manipulated prior to implantation (see, e.g., SURGRAFT dehydrated amniotic sheets, or SURSIGHT ocular amniotic membrane allograft, Surgenex Scottsdale, AZ, PROKERA).

The bio-tissue insert of amniotic membrane can be used alone as a primary treatment or together with an adjunct treatment. The bio-tissue insert of amniotic membrane can be positioned at any of a variety of locations including outside the cornea against the sclera, fornix, bulbar conjunctival surface, cul-de-sac or other part of the eye.

The inserts described herein are preferably formed of biologic material and are self-retained in the eye in a sutureless manner. In some implementations, the inserts are retained without any support or retention aids provided by non-biologic material. Non-biologic material includes synthetic materials prepared through artificial synthesis, processing, or manufacture that may be biologically compatible, but that are not cell-based or tissue-based. For example, non-biologic material includes polymers, copolymers, polymer blends, and plastics. Non-biologic material includes inorganic polymers such as silicone rubber, polysiloxanes, polysilanes, and organic polymers such as polyethylene, polypropylene, polyvinyls, polyimide, etc. The inserts formed of biologic material can be combined with biodegradable or non-biodegradable hardware materials for additional structural stability. The insert formed of biologic material can be cryopreserved, dehydrated, lyopreserved, or non-preserved. The insert can be sterilized prior to use.

Regardless the source or type of biologically-derived material, the material can be cut into a shape and/or formed into an insert suitable for positioning on the ocular surface.

The form factor of the cut inserts can vary as described elsewhere herein. For example, the tissue can be formed into a disk, pellet, plug, or rod shape or a closed loop shape for positioning against the ocular surface to provide release of healing factors from the tissue.

The size and shape of the insert can vary depending on the target location as will be described in more detail below.

The material of the insert can naturally contain a certain level of biologic factors that are relatively homogeneously distributed within the material. The size range of the insert can be selected to ensure wearability and comfort, each of use and handling by clinicians applying the insert to a patient's eye, as well as minimum cubic volume so the insert is capable of eluting biologic factors to the eye within a therapeutic range and/or for biologic activity. The wearability can be provided by a minimum inner diameter of the insert so avoid contact between the insert 100 and the cornea (e.g., 12 mm inner diameter). The wearability can also be provided by a maximum outer diameter that is not too big (e.g., 40 mm outer diameter, preferably about 20 mm-30 mm, more preferably about 22 mm-27 mm) and a width or length that is easy to handle by a clinician placing the insert on the eye, and a width or length that is not too big to induce foreign body sensation. The minimum cubic volume of the insert 100 can be at least about 1mm³, 2 mm³, 3mm³, 4 mm³, 5mm³, 6mm³, 7 mm³, 8 mm³, 9mm³, 10 mm³, 11 mm³, 12 mm³, 13 mm³, 14mm³, 15 mm³, 16 mm³, 17 mm³, 18 mm³, 19 mm³, 20 mm³, 21 mm³, 22 mm³, 23 mm³, 24 mm³, 25 mm³, 50 mm³, 75 mm³, 100 mm³, 125 mm³, 150 mm³ or greater. The minimum cubic volume of the insert 100 can be about 1 mm³ to about 150 mm³, preferably about 14 mm ³.

For inserts in a ring configuration, the difference between the inner diameter and the outer diameter (i.e., width of the insert), as well as the thickness of the insert between the anterior surface to the posterior surface of the insert contributes to the total cubic volume of the insert. The width of the ring provides ease of use to the insert—given that a clinician, such as an ophthalmologist may apply the insert in an operative room or clinic or office setting. A minimum width of about 0.5 mm reliably allows ease of handling and insertion/placement of the insert onto the ocular surface. The minimum width can provide structural rigidity sufficient for manual manipulation by a clinician or other person who is performing the insertion procedure, while also providing a minimal foreign body sensation to the patient. The insert made of amniotic tissue can be positioned on the eye using a finger(s) and with the assistance of a cotton swab to adjust its position under topical anesthesia or with no anesthesia. The insert is applied to the eye so no folding or overlap of the membrane occurs and the lower surface of the membrane lies flush against the anterior surface of the eye. As described elsewhere herein, the size of the ring can vary. The inner diameter of the insert 100 is larger than a patient's cornea so that it encircles with the cornea without touching the cornea, for example, greater than about 9 mm or about 15 mm to accommodate a wider range of corneal sizes. The insert made of amniotic tissue is nearly invisible once positioned on the eye and the tissue blends in with the conjunctiva. The insert can be removed depending on the degree of inflammation in the eye being treated and total time positioned against the eye. The insert may also be completely absorbed within the eye upon positioning for a time.

Processing of chorioamniotic and amniotic membrane tissue for clinical use (e.g., cryopreservation, dehydration, etc.) can lead to variability in thickness that, together with the natural variability in thickness of the tissues, impacts overall cubic volume of the insert formed from the tissue. The insert geometry is designed to achieve a cubic volume that takes into account the natural and acquired variabilities of the tissue during tissue processing. In some implementations, the cubic volume can be within a range of at least about 100mm 3 .

The insert geometry can achieve a cubic volume that falls within a range of at least 1 mm 3 up to about 150mm³. The upper limit can be about 200 micron thickness by an inner diameter of about 12 mm and an outer diameter of about 33 mm.

It should be appreciated that the minimum cubic volume of the insert 100 can be outside this range, but the biologic activity would decrease or increase accordingly. Similarly, if the insert 100 is formed of a dual layer of amnion and chorion, more biologic activity may be observed compared to biologic activity of an insert 100 formed of single layer amnion. A study measuring cytokines in allografts found multilayer amnion/chorion contained similar amounts of each factor measured when normalized per dry weight.

However, single layer amnion allografts contained on average only 25% as much of each factor as the chorion when calculated per surface area of tissue applied to a wound (see Koob et. al., “Cytokines in single layer amnion allografts compared to multilayer amnion/chorion allografts for wound healing” J. Biomed Mater Res B Appl. Biomater. 2015 Jul; 103(5): 1133-40).

While the total volume predicts the overall potential loading of bioactive factors, the precise thickness has variability due to the natural variation in human tissues, both naturally as well as post-processing (e.g., dehydration, cryopreservation, lyophilization, etc.). The pre-processed thickness may also differ from the post-processed, rehydrated thickness, due to changes in the underlying structure of the membrane due to its processing. Therefore, the surface area constructed out of a two-dimensional amniotic membrane sheet, as well as selection of layers (single layer amnion, dual or multilayer amnion, amnionchorion, dual or multilayer amnion-chorion, etc.), will be a determining factor in the overall volume of the construct. In one embodiment, a single layer of amnion-chorion tissue, combined with ring width dimensions described above (a minimum of 1-2 mm in width), with an inner diameter of at least 12 mm (larger than an average cornea), provides adequate structural integrity and rigidity for manual placement of the ring in the eye, as well as adequate volume/surface area for bioactive factor dosing.

The insert can be a single layer of amnion tissue, a multi-stack amnion layer, a single or multi-stack amnion-chorion tissue having an outer diameter that is about 15-35 mm, preferably about 17-33 mm, an inner diameter that is about 12-30 mm, preferably about 16-28 mm, a total area of about 30-1000 mm², a width of about 0.5-6 mm, a thickness of about 0.02-0.5 mm, and a total volume that is about 1-150 mm³, preferably about 5-50 mm³, more preferably about 10-20 mm³.

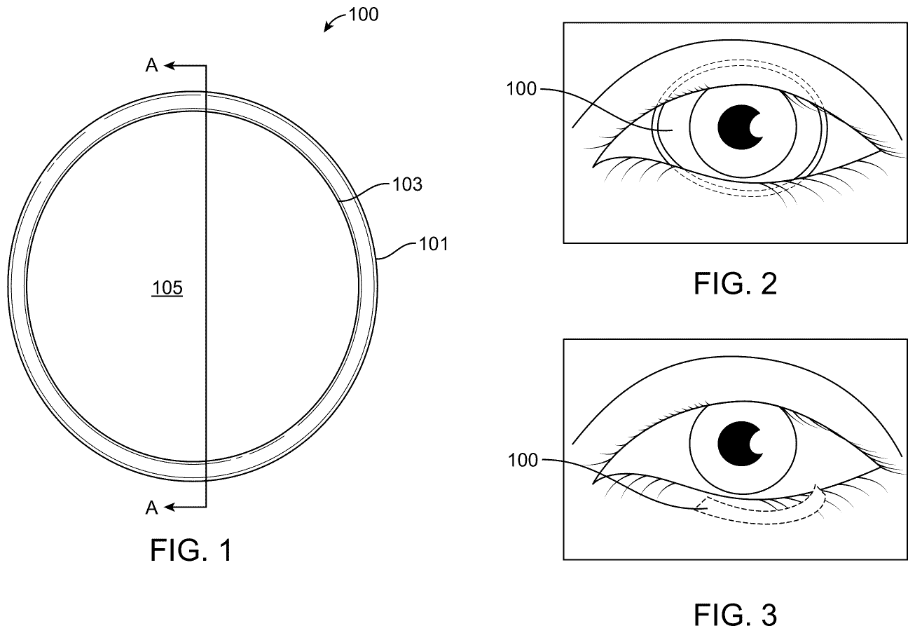

FIG. 1 illustrates an implementation of an insert 100 made of bio-tissue that is configured for self-retaining, sutureless, clear-cornea application to an ocular surface. The insert has an outer edge 101 and an inner edge 103, inside of the outer edge 101. The inner edge 103 defines an opening or central window 105. The central window 105 is sized to extend around a circumference of a cornea to provide for a clear visual axis and to avoid contact with the highly-sensitive corneal surface. The window 105 can be formed by an opening or cutout in the bio-tissue material forming the insert 100 or by forming a closed loop from an elongated strip of material, which is described elsewhere herein. The inner diameter of the insert 100 (i.e., inner diameter of the inner edge 103) is at least about or greater than 9 mm, or at least about 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, and 30 mm. The ocular amniotic peripheral ring can have an outer diameter (i.e., diameter of the outer edge 101) sized so at least a portion of the insert 100 can be positioned on the bulbar conjunctival surface under both eyelids (see FIG. 2). The outer diameter of the insert can vary depending on the inner diameter of the insert. The insert 100 is preferably positioned on a region of the anterior surface of the eye that is outside an optical zone of the eye so that the insert is out of contact with the cornea. Preferably, the outer diameter of the insert is no greater than about 40 mm, 39 mm, 38 mm, 37 mm, 36 mm, 35 mm, 34 mm, 33 mm, 32 mm, 31 mm, 30 mm, 29 mm, 28 mm, 27 mm, 26 mm, 25 mm, 24 mm, 23 mm, 22 mm, 21 mm, and 20 mm and is at least about 12 mm to conform to the bulbar conjunctiva of eye. This outer diameter ensures that the superior part of the insert is retained under the upper eyelid and inferior part of the insert is retained in the cul-de-sac of the lower eye lid, while the medial and lateral sides are retained within the canthi of the eye. In a preferred implementation, the inner diameter of the insert 100 defined by the inner edge 103 is about 20 mm and the outer diameter of the insert 100 defined by the outer edge 101 is no greater than about 30 mm.

In an implementation, the insert 100 has an inner diameter of about 20-22 mm, an outer diameter of about 24-27 mm, a thickness between upper and lower surfaces that is about 0.05-0.150 mm, and a volume that is about 10-18 mm³. Thickness of the insert can vary due to anatomy, however, a nominal thickness of 0.1 mm provides a volume of about 14 mm³. The dimensions of this implementation provide a comfortable fit for most eyes and ensures the insert avoids touching the cornea, and avoids buckling or folding, such as at the lateral canthus, which can impact retention and comfort. The dimensions of this implementation also make the insert easy for handling by the clinician applying the insert to the ocular surface, which can be applied manually and adjusted with a cotton swab or similar instrument. The dimensions of this implementation also provide a total surface area/volume sufficient to achieve bioactive factor dose to the patient from the insert 100 for anti-fibrotic, anti-inflammatory, or anti-neovascular therapeutic effect while positioned on the eye surface.

The insert can have a width between the outer edge 101 and the inner edge 103.

The width can be at least about 100 microns and no greater than about 8 mm, no greater than about 7 mm, no greater than about 6 mm, no greater than about 5 mm, no greater than about 4 mm, no greater than about 3 mm, no greater than about 2 mm, or no greater than about 1 mm. Preferably, the width between the outer edge 101 and the inner edge 103 is about 0.5 mm up to about 16 mm, preferably 1 mm up to about 5 mm. The total area of the annulus formed by the insert can be about 30 mm² up to about 1000mm² , preferably about 50 mm² to about 200 mm².

The insert can have a thickness between the lower surface of the ring (i.e., the portion contacting the anterior surface of the eye) and the upper surface that is at least 15 microns up to about 800 microns, preferably the thickness is about 20-200 microns depending on whether the implant is a single layer amnion, multi-layer amnion, single layer amniochorion or multi-layer amniochorion.

In other implementations, the minimal structural modification of the biological tissue includes cutting the tissue into a first shape and then forming the insert into a second shape. In some implementations, the first shape is a disc shape and the second shape is a closed loop. The disc shape can have an outer diameter as described above, for example, no greater than about 40 mm or another size that is shaped to fit on a surface of an eye. The disc can be cut to have an inner diameter sized large enough to ensure the bio-tissue can remain out of contact with the cornea (e.g., at least about 8-9 mm). The disc shape can be a regular geometric shape (e.g., circular, oval, elliptical, polygonal, and the like) or the disc shape can be a more irregular shape (e.g., fusiform) that is then cut into an annular shape as discussed above. The geometry of the insert 100 can vary. In some implementations, the insert 100 includes a connected region forming a closed loop having any of a variety of shapes, including round, such as a circular, oval, elliptical shape. The geometry of the closed loop can also include serpentine, saddle shape, toric, or polygonal having n number of sides. The geometry may be a polygon, including but not limited to a rectangle, pentagon, hexagon, octagon, etc. The geometry may be a combination of circular and straight segments, such as a half-circle. The geometry may include a variable width along the circumference of the insert, including but not limited to flaps, pads, wings, in order to increase total surface area/volume and thereby increase the bioactive factor dose provided by the insert. These features may particularly be on the superior or inferior portions of the insert, resting underneath the eyelid, to avoid the lid margins or the region where the eyelids meet when the eye closes or blinks.

The minimal structural modification of the tissue can adjust a size of the tissue to form an insert 100 having a form factor that is an elongate pellet, rod, strip, ring segment or other elongate insert sized and shaped for positioning on an ocular surface, such as under one or both eye lids. In this manner, the bio-tissue insert 100 need not form a closed loop shape and instead can be positioned on the eye out of contact with the cornea, but as an elongate strip or segment. FIG. 3 illustrates an ocular amniotic peripheral insert segment positioned on the bulbar surface under the lower eye lid. The insert can have an outer edge, an inner edge, a first free end, and a second free end opposite the first. The insert can have length between the first free end and the second free end, a width between the outer edge and the inner edge, and a thickness. The length can be about 5 mm and preferably no greater than about 33 mm. The width can be about 1 mm, preferably no greater than about 100 mm. The thickness, which can be the thickness of the starting biological material (i.e., amniotic membrane), can be no greater than about 5 mm. Amniotic membrane is typically between 0.02 mm-0.05 mm. In an implementation, the insert 100 can include one or more ring segments, strips or elongated inserts that are not wider than 10 mm and longer than 30 mm. The insert 100 can be inserted in the cul-de-sac of the lower lid or underneath the upper lid on the sub-palpebral ocular surface, or both.

Creating a ring shape from a disc shape of biologic tissue generates a large surface area of tissue that remains unused and thereby increases the cost to manufacture the insert. In some implementations, the minimal structural modification of the biological tissue (e.g., amniotic membrane) can include a longitudinal cutting into an elongate strip of tissue that is then formed into another shape, such as a closed loop shape. The elongate strip, for example, can be sized so that upon forming a closed loop forms an insert 100 having an inner diameter as described above. Generally, the elongate strip of tissue has a width that is less than its length.

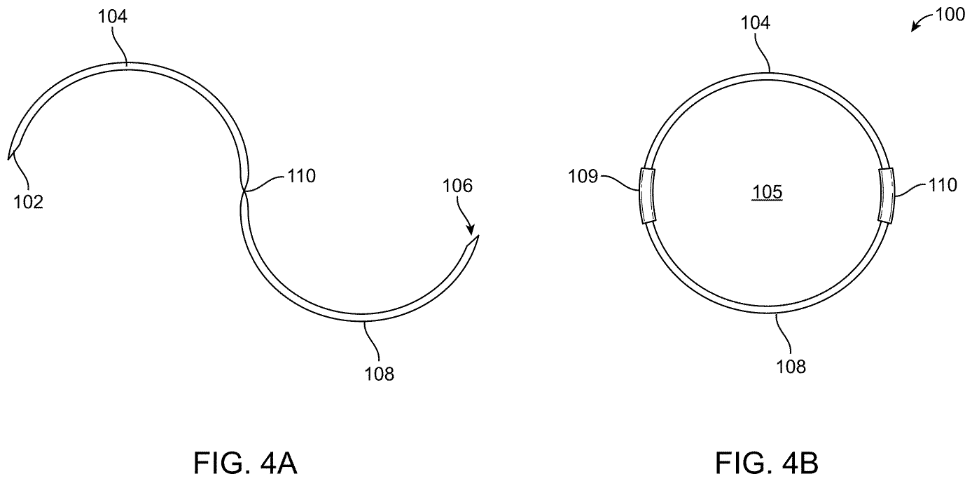

Manufacturing the inserts using bio-tissue cut into strips that are later fused into a closed loop ensures nearly all tissue is used with less waste. FIG. 4A illustrates an initial cut form factor of bio-tissue having a thin sine wave shape. The strip of bio-tissue has a first segment 104 having a first free end 102. The first segment 104 has an arc shape. The strip of bio-tissue has a second segment 108 having a second free end 106. The second segment 108 has an arc shape corresponding to the shape of the first segment 104. The first segment 104 and the second segment 108 are twisted relative to one another at a hinge point 110 that is about half-way between the first free end 102 and the second free send 106. The arc shapes and the twisted hinge point 110 creates a sine wave shape of the initial cut form factor. FIG. 4B illustrates the bio-tissue insert 100 formed by fusing the first free end 102 and the second free end 106 together at a first joint 109 forming a closed loop. The hinge point 110 where the segments 104, 108 are twisted can be fixed forming a second joint 111. The joints can be formed by a variety of fixation methods, including a polymer coating, adhesive, or other material. The fixation can alternatively be done without any additional materials, but using the processing of the amniotic membrane itself, e.g., cutting the tissue in its wet, unprocessed form, then clamping the structure in place during processing (e.g., dehydration, cryopreservation, lyophilization, etc.). The first joint 109 and the hinge point 110 ensure the insert 100 maintains the closed loop and the arc shapes of the segments 104, 108 result in a substantially circular shape to the insert 100.

The joint 109 can be formed by adhering the ends, such as by wetting dehydrated tissue allowing them to stick together. In some implementations, the joint 109 is formed by mechanically linking the first and second free ends 102, 106 together. For example, each of the free ends can have a partial slit formed through its width that is sized to receive the partial slit of the opposite end.

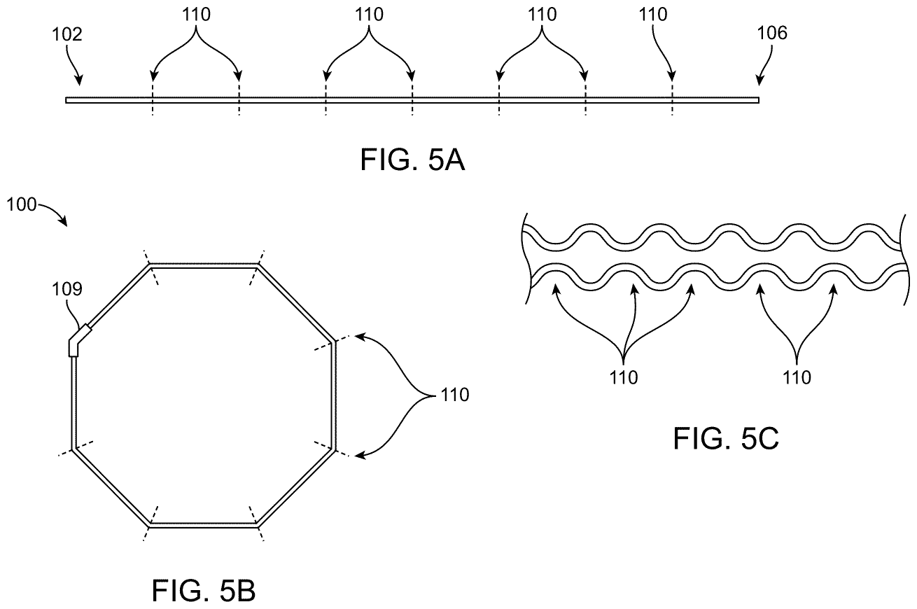

The closed loop insert need not have a smooth circular shape as shown in FIG. 1 or FIG. 4B. The insert 100 can have a closed shape having n sides forming a polygon. FIG. 5A illustrates an initial cut form factor of bio-tissue having a thin elongate shape. The strip of bio-tissue has a first free end 102 and a second free end 106. FIG. 5B illustrates the bio-tissue insert 100 formed by folding the strip of FIG. 5A at multiple hinge points 110 along its length and fusing the first free end 102 and the second free end 106 together at a single joint 109 forming a closed loop. Unlike the closed loop shape of FIG. 4B, the closed loop shape of FIG. 5B incorporates multiple sides forming a polygonal closed loop. The number and angle of the hinge points 110 can vary. The hinge points 110 can have an angle that varies depending on the closed shape having n sides. The insert 100 shown in FIG. 5B is created by folding the strip of FIG. 5A at 8 hinge points 110 thereby forming an 8-sided polygon (i.e., octagon). Thus, the angle between sides at each hinge point 110 is 135 degrees.

The hinge points 110 can incorporate features to facilitate folding of the bio-tissue strip, such as slits in the bio-tissue at the desired location of the hinge points 110. The bio-tissue can incorporate a narrower width at the desired hinge points 110 to encourage folding. The strip of bio-tissue need not be straight along its entire length prior to folding and can instead be cut into an elongate strip having undulating edges (see FIG. 5C). The undulating edges can aid in folding the elongate strip into a closed loop having a smoother shape. The pattern of the undulations on each edge can mirror one another such that each edge has a smoothly falling form towards its opposing edge creating a narrowing region for the hinge points 110 and a smoothly rising form away from one another creating a widening region between the hinge points 110 (see FIG. 5C). In still further implementations, the bio-tissue can be formed into another three-dimensional shape such as a ribbon shape that is then wrapped around a cylinder or cone to flex the bio-tissue insert before being placed onto the eye. Whether the bio-tissue is cut into an annulus, an S-shaped strip, a straight strip, or an undulating strip, the bio-tissue insert is prepared in a minimally-modified manner with cutting, shaping, folding, bending, and fusing, etc.

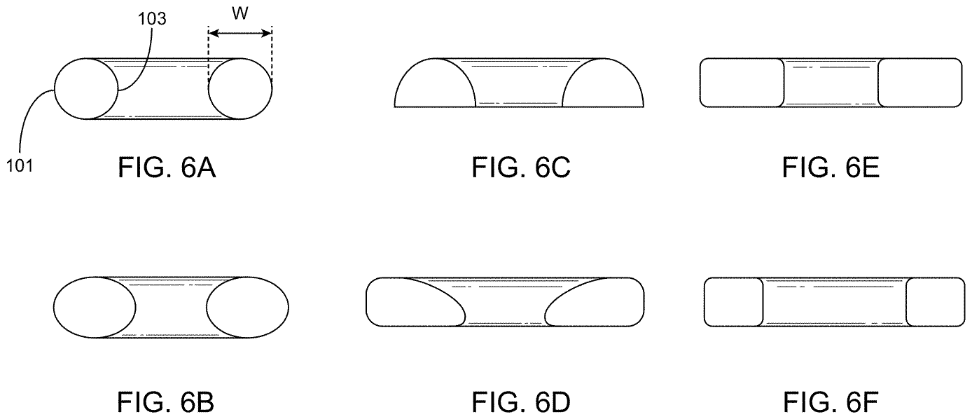

The cross-section of the bio-tissue insert 100 (i.e., cross-section taken from a surface of the tissue configured to be placed against the eye to a surface of the tissue that is configured to face away from the surface) can have different shapes. The shape of the cross-section can be rounded or angular. For example, the rounded cross-sectional shape can include a circle, oval, ellipse, hemi-circle, and the like. The angular cross-sectional shape can include rectangular or square shapes having at least one flat surface, akin to a flat sheet. The cross-section of the insert 100 can have a width W between the inner edge 103 and the outer edge 101 that is at least about 0.1 mm to about 10 mm, preferably about 1 mm up to about 5 mm. FIGS. 6A-6F illustrate example cross-sections of the insert 100 of FIG. 1 taken at line A-A including circular (FIG. 6A), oval (FIG. 6B), hemi-spherical (FIG. 6C), and tear-drop shaped (FIG. 6D), rectangular (FIG. 6E), square (FIG. 6F) and the like.

Upon positioning the insert 100 onto the eye, the bio-tissue of the insert 100 can absorb fluids of the eye and rehydrate. Such fluids can be topically delivered medications (e.g., eye drops) which will be absorbed and retained into the amniotic biotissue which can then serve as a sustained release depot to increase the bio-availability of the medications to the ocular surface.

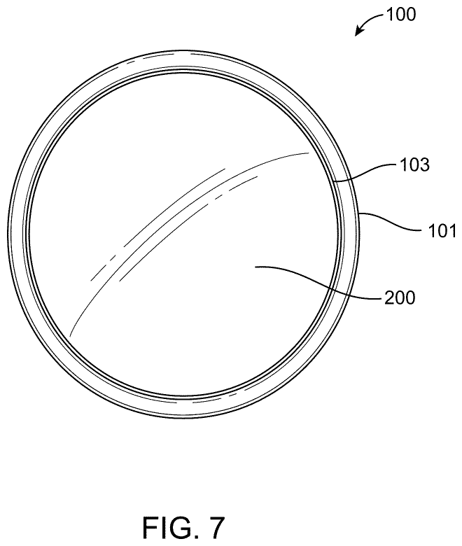

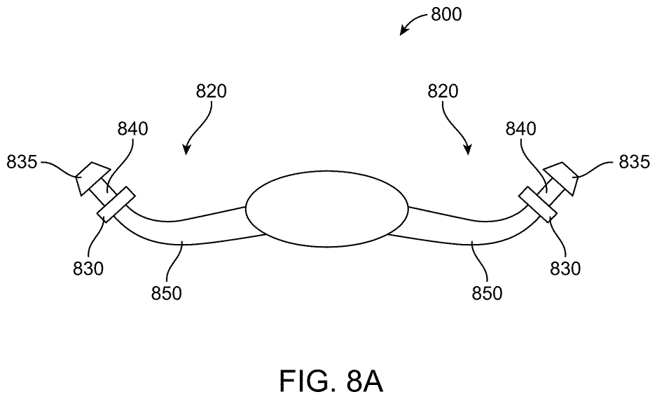

In some implementations, the insert 100 can incorporate a separate retention feature 200 configured to provide structural stability to the insert. The configuration of the retention feature 200 can vary, including a separate anchor or other retaining structure, a bandage, or standard corneal contact lens. FIG. 7 shows an insert 100 coupled to a retention feature 200 that is a corneal contact lens having an outer perimeter sized to be received within the opening 105 of the insert 100 so that the insert 100 conforms to the outer perimeter of the corneal contact lens. The inner edge 103 of the insert 100 is greater than a diameter of the outer perimeter of the retention feature 200. Thus, the peripheral edge of the corneal contact lens serves as a retaining element to the insert 100 to keep the insert 100 centered on the anterior surface of the eye. The amniotic membrane insert 100 can be the same thickness or thinner than the retention feature 200. The insert 100 thus encircles but does not snag the palpebral surface. The window 105 of the insert 100 can have a diameter that is 0.1-2 mm greater than the diameter of the outer perimeter of the retention feature 200. The insert 100 can have a thickness that is less than 4 mm and/or thinner than a thickness of the retention feature 200. The retention feature 200 can remain in place on the eye as long as the insert 100 remains in place. The retention feature 200 can biodegrade before or after the insert 100 is no longer positioned on the eye. Additionally, the insert inner edge can have a larger diameter than the cornea and the retention feature can be completely transparent resulting in no obstruction of vision while still allowing the amniotic membrane to release the bioactive factors into the tear film.

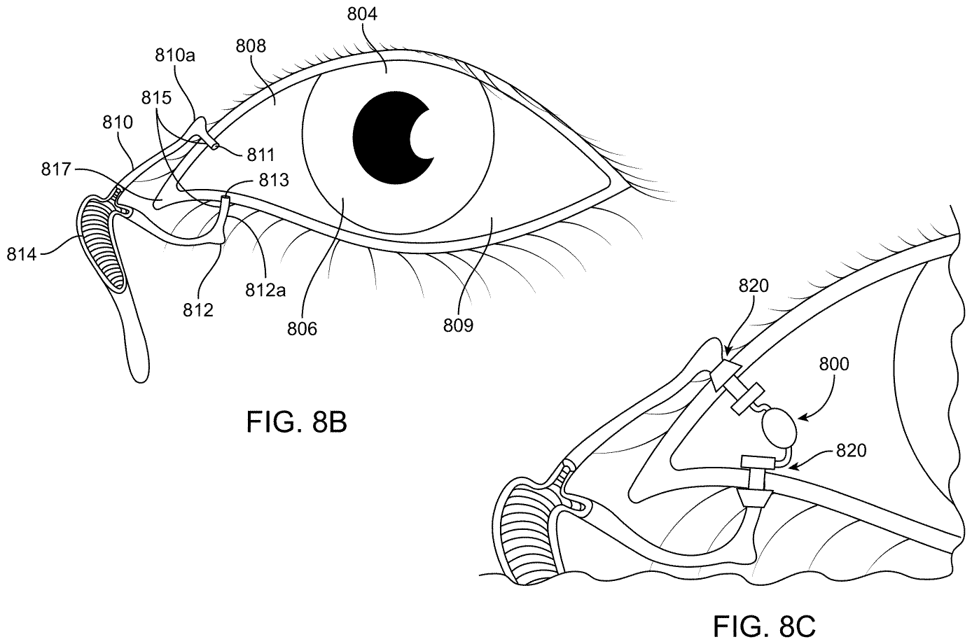

The biotissue insert having a separate retention feature need not be annular in shape nor encircle the globe of the eye and the retention feature need not cover or contact the cornea. FIG. 8A shows a biotissue insert 800 coupled to at least one retention feature 820.

The insert 800 can be a segment of amnion, chorion, amnio-chorion as described elsewhere herein. The insert 800 can have any of a variety of shapes and need not be annular, including a strip, disc, or other segment of tissue sized to contact at least a portion of the ocular surface. The retention feature 820 can be in the form of a punctal plug coupled to the insert 800 by a tissue connector 850. The insert 800 is preferably positioned using a pair of retention features 820, each of the retention features 820 being in the shape of a punctal plug and arranged to insert in each of the upper and lower punctum (see FIG. 8C).

FIG. 8B shows an eye including a cornea 804, an iris 806, and a sclera 808 surrounding the cornea 804 and iris 806. A conjunctival layer 809 is substantially transparent and disposed over sclera 808. The lacrimal system includes an upper canaliculus 810 and a lower canaliculus 812, collectively the canaliculae, and the nasolacrimal duct or sac 814. The upper and lower canaliculae 810, 812 terminate in an upper punctum 811 and a lower punctum 813, respectively, which are also referred to as punctal apertures. The punctal apertures 811, 813 are situated on a slight elevation at the medial end of the lid margin at the junction 815 of the ciliary and lacrimal portions near the medial canthus 817. The punctal apertures 811, 813 are round or slightly ovoid openings surrounded by a connective ring of tissue. Each of the punctal openings 811, 813 leads into a vertical portion 810a, 812a of the respective canaliculus before turning horizontally to join its other canaliculus at the entrance of a lacrimal sac 814. The canaliculae 810, 812 are tubular and lined by stratified squamous epithelium surrounded by elastic tissue which permits the canaliculus to be dilated.

The punctal plug retention feature 820 is shaped to insert at least in part within one of the punctal apertures 811, 813 so that the insert 800 is positioned on a surface of the eye near the puncta (see FIG. 8C). The retention feature 820 can include a first punctal plug coupled to the insert 800 by a first tissue connector 850, the first punctal plug being positioned within a first puncta, and a second punctal plug coupled to the insert 800 by a second tissue connector 850, the second punctal plug being positioned within a second puncta. The biotissue insert 800 can be coupled to each of the first and second punctal plugs so that the insert 800 spans generally between the retention features 820.

The retention feature 820 having a shape of a punctal plug can incorporate a collar 830 at a proximal end, a tapered portion 835 near a distal end, and a body portion 840 extending between the collar 830 and the tapered portion 835. The tapered portion 835 is sized and shaped to project into the canaliculus 810, 812. The taper angle of the tapered portion 835 eases insertion of the tapered portion into the punctum 811, 813. The tapered portion 835 can have a proximal ledge feature 840 that is designed to prevent the plug from being inadvertently dislodged (i.e., withdrawn) from the canaliculus. The collar 830, in use, is sized and shaped to rest against the exterior of a punctum 811, 813. The collar 830 has a diameter sized to prevent the collar 830 from entering the canaliculus. It is also designed to minimize irritation to the eye. The body portion 840 extending between the collar 830 and the tapered portion 835 is sized and shaped to reside comfortably within the canaliculus 810, 812. The collar 830, tapered portion 835, and body portion 840 can each be formed from a flexible material, such as silicone, that is impermeable to fluids. The material is biocompatible and sufficiently flexible to mold into shape within the canaliculus. The tissue connector 850 can project proximally from the collar 830 (i.e., away from the distal end of the tapered portion 835 located within the canaliculus). The tissue connector 850 couples the biotissue insert 800 to a region of the collar 830 or other portion of the plug that remains outside the punctum so that the insert 800 is fixed relative to a surface of the eye up insertion of the body portion 840 into the canaliculus 810, 812. The tissue connector 850 can be formed of any of a variety of materials, including suture, molded silicone, or other biocompatible material. The tissue connector 850 can be connected to the plug with adhesive or by a mechanical clamp or by molding onto the tissue, with a feature in the amniotic membrane such as a hole or holes that mate with features on the tissue connector). At least one punctal plug or each plug as well as the tissue connector can be constructed of amniotic membrane. In one implementation, the entire plug, tissue connector, and insert construct is composed of a single continuous sheet of amniotic membrane that is cut and processed into the shape.

The body portion 840 can, but need not, be tubular and extend between two apertures allowing fluid flow through the plug. In preferred implementations, the body portion 840 is solid and the plug includes no apertures so that no fluid flows through the retention feature 820 so that the plug prevents tear film drainage. Punctal occlusion prevents drainage of the tear film through the lacrimal system thereby enhancing tear film preservation, which is particularly useful for treating dry eye. The increased tear film also improves bioelution from the biotissue insert 800, which is positioned in contact with the medial ocular surface around or on top of the caruncle. The biotissue insert 800 can be positioned under the lid within the cul-de-sac of the eye and retained by a single punctal plug retention feature 820. Bi-canalicular cross-punctal fixation of a biotissue insert provides sutureless, self-retaining anchoring of the device on the ocular surface and provides the benefit of punctal occlusion and ocular surface bio-elution.

EXPERIMENTAL Initial studies were conducted to assess tolerance of the amniotic tissue insert in patients. The insert made of amniotic tissue cut into a ring shape having a 24-mm inner diameter and 27-mm outer diameter. Other insert sizes evaluated had 21-mm inner diameter and 25-mm outer diameter. The insert was positioned on the anterior surface of the eye, with or without topical anesthesia, using fingers and a cotton swab to adjust positioning. The inserts were well tolerated by patients, even those without prior experience using contact lenses or glasses. Patients were unable to sense the presence of the insert by about 5 minutes post-placement. In some instances where the insert folded slightly at the lateral canthus, patients experienced sensation of the insert that resolved upon flattening the lower surface of the insert against the eye. The insert was nearly invisible on the anterior surface of the eye due to it blending into the conjunctiva. The inserts were easily removed after 30 minutes with fingers. The inserts were retained without any inadvertent slippage or folding.

In various implementations, description is made with reference to the figures.

However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, ”an implementation,“ or the like, means that a particular feature, structure, configuration, or characteristic described is included in at least one embodiment or implementation. Thus, the appearance of the phrase ”one embodiment,“ ”an embodiment,“ ”one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

1.-13. (canceled)

14. An insert made of amniotic membrane that is configured for self-retaining, sutureless, clear-cornea application to an ocular surface, the insert having an outer edge, an inner edge, a first free end, and a second free end opposite the first free end, wherein a length of the insert between the first free end and the second free end and a width of the insert between the outer edge and the inner edge is sized to be received within the cul-de-sac of an eyelid.

15. The insert of claim 14, wherein the eyelid is an upper eyelid or a lower eyelid.

16. The insert of claim 14, wherein the length of the insert is no greater than 33 mm, the width of the insert is no greater than 100 mm, and a thickness of the amniotic membrane is no greater than 5 mm.

17. The insert of claim 14, further comprising a retention feature separate from the insert and configured to provide structural stability to the insert.

18. The insert of claim 17, wherein the retention feature is formed of a biodegradable or non-biodegradable material.

19. The insert of claim 14, wherein the amniotic membrane includes amnion membrane.

20. The insert of claim 19, wherein the amniotic membrane includes amnion membrane and chorion layer.

21.-23. (canceled)

24. An insert of amniotic membrane coupled to a punctal plug, the punctal plug sized and shaped to insert at least in part within a punctum of an eye to position the amniotic membrane in contact with a surface of the eye.

25. The insert of claim 24, wherein the amniotic membrane is shaped into an elongate strip or disk.

26. The insert of claim 24, wherein the punctal plug is solid so as to occlude the punctum and reduce drainage of fluid out of the eye.

27. The insert of claim 24, wherein, upon positioning the insert in the eye, the amniotic membrane is positioned under a lid of the eye.

28. The insert of claim 27, wherein the amniotic membrane is positionable within the cul-de-sac of the eye.

29. The insert of claim 24, wherein the punctal plug comprises a proximal collar, a distal tapered region, and a body extending between the collar and the tapered region.

30. The insert of claim 29, wherein the amniotic membrane is coupled to the collar of the punctal plug by a tissue connector.

31. The insert of claim 24, further comprising a second punctal plug coupled to the amniotic membrane.

32. The insert of claim 31, wherein the punctal plug and/or the tissue connector are amniotic membrane.

33.-36. (canceled)

37. An implant made of amniotic membrane that has a size and a shape to insert at least in part within a punctal aperture of an eye.

38. The implant of claim 37, wherein the amniotic membrane is a single continuous sheet cut and processed into the shape, the shape being an elongate strip.

39. The implant of claim 37, wherein the implant is self-retaining and solid so as to occlude the punctal aperture to decrease tear film drainage through the punctal aperture.

40. The implant of claim 37, wherein the amniotic membrane includes amnion membrane alone or amnion membrane and chorion layer.
